(19) United States
(12) Patent Application Publication
(10) Pub. No.: US 2001/0019832 A1
(43) Pub. Date: Sep. 6, 2001

(54) Methods for identification using U0126

(76) Inventor: Marguerite Luthman, Lidingo (SE)

Correspondence Address: DINSMORE & SHOHL, LLP, 1900 CHEMED CENTER, 255 EAST FIFTH STREET, CINCINNATI, OH, 45202, US

(21) Appl. No.: 09/797,227

(22) Filed: Mar. 1, 2001

Related U.S. Application Data
(60) Provisional application No. 60/188,550, filed on Mar. 10, 2000.

(30) Foreign Application Priority Data
Mar. 3, 2000 (SE) 0000718-7

(51) Int. Cl. C12Q 1/54

(52) U.S. Cl. 435/14; 435/15

(57) ABSTRACT

The present invention relates to a method for identifying an agent useful for the treatment or prevention of a medical condition related to reduced cellular uptake of glucose, said method comprising the steps of:

(i) identifying a target polypeptide interacting with the compound U0126 (1,4-diamino-2,3-dicyano-1,4-bis [2-aminophenylthio] butadiene);

(ii) contacting a candidate agent with the said target polypeptide; and

(iii) determining whether said candidate agent modulates the biological activities of the said target polypeptide, such modulation being indicative for an agent useful for the treatment or prevention of a medical condition related to reduced cellular uptake of glucose.

### TECHNICAL FIELD

The present invention relates to the use of 1,4-diamino-2,3-dicyano-1,4-bis [2-aminophenylthio] butadiene (U0126) in methods for identification of agents useful in the treatment of medical conditions related to reduced cellular uptake of glucose, in particular type II diabetes.

### BACKGROUND ART

One of the major hormones that influence metabolism is insulin, which is synthesized in the beta cells of the islets of Langerhans of the pancreas. Insulin primarily regulates the direction of metabolism, shifting many processes toward the storage of substrates and away from their degradation. Insulin acts to increase the transport of glucose and amino acids as well as key minerals such as potassium, magnesium, and phosphate from the blood into cells. It also regulates a variety of enzymatic reactions within the cells, all of which have a common overall direction, namely the synthesis of large molecules from small units. A deficiency in the action of insulin (diabetes mellitus) causes severe impairment in (i) the storage of glucose in the form of glycogen and the oxidation of glucose for energy; (ii) the synthesis and storage of fat from fatty acids and their precursors and the completion of fatty-acid oxidation: and (iii) the synthesis of proteins from amino acids.

There are two varieties of diabetes. Type I is insulin-dependent diabetes mellitus (IDDM), for which insulin injection is required; it was formerly referred to as juvenile onset diabetes. In this type, insulin is not secreted by the pancreas and hence must be taken by injection. Type II, diabetes, non-insulin-dependent diabetes mellitus (NIDDM), is characterized clinically by hyperglycemia and insulin resistance and is commonly associated with obesity. Type II diabetes is a heterogeneous group of disorders in which hyperglycemia results from both an impaired insulin secretory response to glucose and decreased insulin effectiveness in stimulating glucose uptake by skeletal muscle and in restraining hepatic glucose production (insulin resistance). Before diabetes develops, patients generally loose the early insulin secretory response to glucose and may secrete relatively large amounts of proinsulin. In established diabetes, although fasting plasma insulin levels may be normal or even increased in type II diabetes patients, glucose-stimulated insulin secretion is clearly decreased. The decreased insulin levels reduce insulin-mediated glucose uptake and fail to restrain hepatic glucose production.

Glucose homeostasis depends upon a balance between glucose production by the liver and glucose utilization by insulin-dependent tissues, such as fat and muscle, and insulin-independent tissues, such as brain and kidney. In type II diabetes, the entry of glucose into fat and muscle is reduced and glucose production in the liver is increased, due to insulin resistance in the tissues.

The primary role of insulin is as regulator of glucose homeostasis, but in addition insulin also acts as a stimulator of cell growth and protein synthesis. Binding of insulin to the insulin receptor (IR) and phosphorylation of the insulin receptor substrates ½ (IRS½) initiate the two signal transduction events, glucose homeostasis and the mitogenic pathway (see FIG. 1). Signaling via phosphoinositide 3-kinase (PI 3-K) is crucial for insulin-stimulated glucose uptake. Ras, the signaling molecule upstream the mitogen-activated protein kinase (MAPK), mediates the mitogenic action of insulin. The two kinases, MEK1 and MEK2, phosphorylate the extracellular signal-regulated kinases 1 and 2 (ERK½). These kinases in the MAPK pathway play a pivotal role in mediation of cellular responses to a variety of signaling molecules, e.g. growth and differentiation factors, from the plasma membrane receptors to the nucleus leading to gene transcription and protein synthesis. For a review, see e.g. White, M. F., “Protein Tyrosine Kinases in Signal Transduction” in: MCR 2000 Syllabus, The Endocrine Society (ISBN 1-879225-37-9).

Wortrmannin (an antibiotic isolated from Penicillium wortmanni) is a compound known to inhibit insulin-induced signaling in the carbohydrate pathway at the level of PI 3-kinase (cf. FIG. 1). A cross-talk between PI3-K in the carbohydrate pathway and MEK/ERK in the mitogenic pathway has been identified. For instance, wortmannin has been shown to increase the MEK/ERK signaling in the mitogen-activated cascade.

A commonly used inhibitor for the MAPK signaling pathway is PD98059 (2-Amino-3-methoxyflavone), which inhibits activation (phosphorylation) of inactive MEK½ (cf. FIG. 1). The compound U0126 (1,4-diamino-2.3-dicyano-1,4-bis [2-aminophenylthio] butadiene) has recently been shown to be a MAPK inhibitor (Favata M F et al. (1998) J. Biol. Chem. 273:18623-18632; Goueli, S. A. et al. (1998) Promega Notes 69, p.6). U0126 specifically inhibits MEK½ in both the inactive and active forms. This is in contrast to PD98059, which only inhibits phosphorylation of inactive MEK½ (Favata et al., supra; cf. FIG. 1). For a review of protein kinase inhibitors, see e.g. Davies, S. P. et al. (2000) Biochem. J. 351: 95-105.

The compound U0126 has been identified as an inhibitor of AP-1 transactivation in a cell-based reporter assay used to screen for an anti-inflammatory drugs (Duncia, J. V. et al. (1998) Bioorganic & Medicinal Chemistry Letters 8: 2839-2844). Antagonism of the transcription factor AP-1 and NF-kappa B, which are important regulators of immune response genes, has been demonstrated to be the primary mechanism for the anti-inflammatory and immune suppressive effects of steroids U0126 was also shown to inhibit, in fibroblasts, genes containing AP-1 response element in their promoters. These effects of U0126 result from direct inhibition of MEK½. Activation of this pathway regulates the activity of a number of substrates through phosphorylation. These substrates include transcription factors such as Elk-I, c-Myc, ATF2 and the AP-1 components, c-Fos and c-Jun. Thus, inhibition of MEK½ by U0126 leads to a decreased expression of the downstream targets c-jun and c-fos and inhibition of AP-1 transactivation. Despite the difference in site of action of U0126 and PD98059, both substances inhibit c-fos production and further block the AP-1 transactivation.

### BRIEF DESCRIPTION OF THE DRAWINGS

FIG. 1: Scheme of the insulin signaling pathways leading to glucose uptake and protein synthesis. Black bars indicate inhibitory activities. For further details, see the background art section.

FIG. 2: Glucose uptake rate in rat skeletal muscle cells.

FIG. 3: Glucose uptake rate in human skeletal muscle cells.

FIG. 4: Glucose uptake rate in human neuroblastoma cells. “Wo” is abbreviation for wortmannin.

FIG. 5: Glucose oxidation in rat skeletal muscle cells.

FIG. 6: Palmitate oxidation in rat skeletal muscle cells during insulin resistant conditions.

FIG. 7: PKB activity in rat skeletal muscle cells.

FIG. 8: Insulin secretion in rat insulinoma cells.

### DISCLOSURE OF THE INVENTION

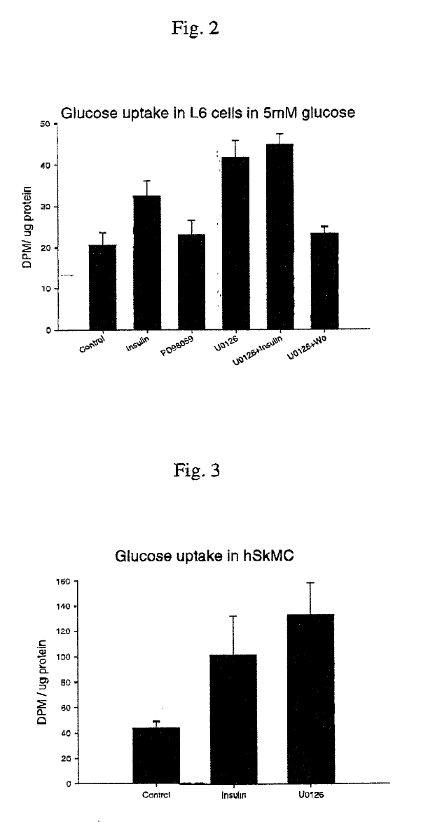

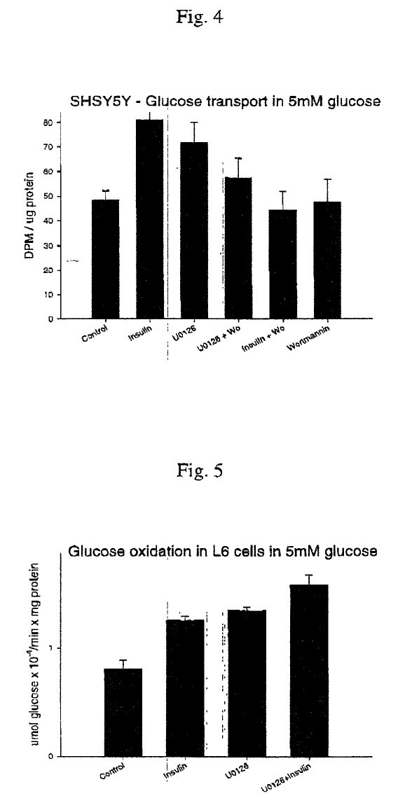

It has surprisingly been found that the compound 1,4-diamino-2,3-dicyano-1,4-bis [2-aminophenylthio] butadiene (also referred to as U0126), controls glucose homeostasis in cell lines from different species. U0126 increases glucose transport and glucose oxidation and decreases fatty acid oxidation in the rat skeletal muscle cell line L6 during normal and insulin resistant conditions (FIGS. 2, 5 and 6). U0126 also increases glucose transport in human skeletal muscle cells (FIG. 3) and in a human neuroblastoma cell line, SH-SY5Y (FIG. 4). Furthermore, U0126 stimulates insulin secretion in a rat insulinoma cell line. INS-1 (FIG. 8).

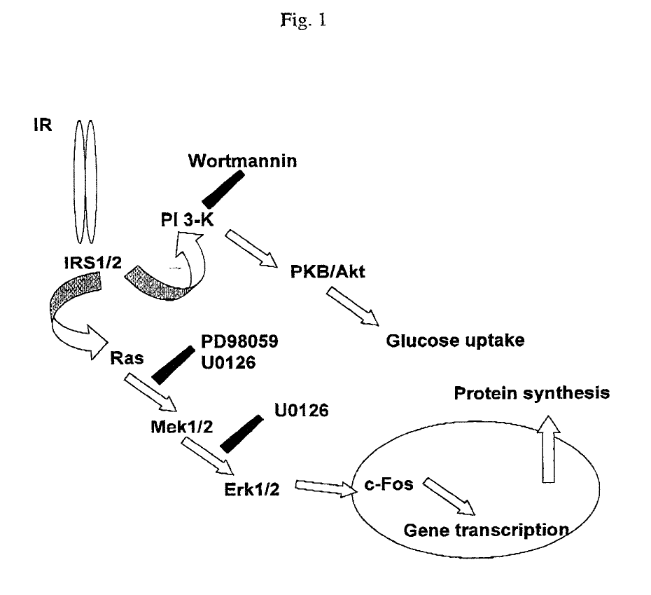

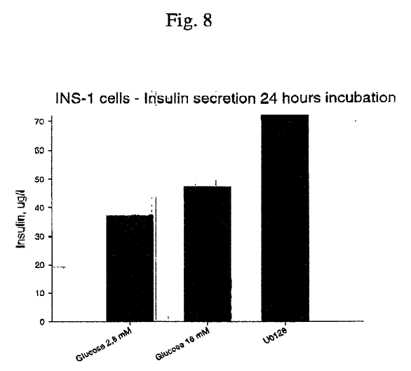

The insulin-like effect of U0126 in L6 cells was not mediated by activation of PKB/Akt (FIG. 7). However, the PI 3-kinase inhibitor wortmannin inhibited the increase in glucose uptake caused by U0126 (FIG. 4), indicating that U0126 affected at least PI 3-kinase (cf. FIG. 1). There was no additive effect of U0126 when glucose uptake was induced by insulin together with U0126 in L6 cells (FIG. 2), indicating that some components in the PI 3-kinase signaling pathway is shared. Interestingly, in contrast to insulin. U0126 showed insulin-like effects also during insulin resistant conditions. The effects of U0126 on carbohydrate homeostasis were not mediated by the previously known activity of U0126 (i.e. inhibition of the MAPK signaling pathway) since the other known inhibitor of the MAPK pathway, PD98059 (2-Amino-3-methoxyflavone), did not show these effects.

The present invention thus relates to the use of 1,4-diamino-2,3-dicyano-1,4-bis [2-aminophenylthio] butadiene (U0126) in methods for identification of agents useful in the treatment of medical conditions related to reduced cellular uptake of glucose, in particular type II diabetes.

This invention also provides methods for identifying therapeutically useful agents having similar properties as U0126. Such agents may be identified by reacting a target polypeptide, with a substance which potentially binds to the said target polypeptide, and assaying for substance-polypeptide complex, for free substance or for non-complexed target polypeptide, or for activation of the target polypeptide. The substance-polypeptide complex free substance or non-complexed target polypeptides may be isolated by conventional isolation techniques, e.g. salting out, chromatography, electrophoresis, gel filtration, fractionation, absorption, agglutination, or combinations thereof.

In particular, this invention provides a method for identifying an agent useful for the treatment or prevention of a medical condition related to reduced cellular uptake of glucose, said method comprising the steps of:

(i) identifying a target polypeptide interacting with the compound U0126;

(ii) contacting a candidate agent with the said target polypeptide, under conditions which permit binding of the candidate agent and the target polypeptide; and

(iii) determining whether said candidate agent modulates the biological activities of the said target polypeptide, such modulation being indicative for an agent useful for the treatment or prevention of a medical condition related to reduced cellular uptake of glucose.

As used herein, the term “agent” means a biological or chemical compound such as a simple or complex organic molecule, a peptide, or a protein.

The said medical condition related to reduced cellular uptake of glucose is, in particular, type II diabetes.

The said target polypeptide can be a kinase, in particular a kinase involved in the insulin signaling pathway, such as phosphoinositide 3-kinase (PI 3-kinase). However, the skilled person will be able to determine whether U0126 binds to other polypeptides that are suitable for use in screening assays. The interaction between the target polypeptide and the compound U0126 can be determined by methods known in the art. For instance, labeled U0126 can be used to determine binding to polypeptides involved in glucose metabolism and insulin signaling. Where a radioactive label is used as a detectable substance, the target polypeptide may be localized by autoradiography.

In the method according to the invention, the compound U0126 can conveniently be used as a control substance for determining the effect of the candidate agent. In such a comparison, an effect similar to the effects of U0126 would be indicative that the candidate agent is useful for the treatment or prevention of a medical condition related to reduced cellular uptake of glucose. Suitable methods, such as competition assays are known in the art, see e.g. Lutz, M. & Kenakin, T. “Quantitative Molecular Pharmacology and Informatics in Drug Discovery”, John Wiley & Sons Ltd., 1999.

In a further aspect, the invention provides the use of an agent, identified by the methods described above, in methods for treatment of a medical condition related to reduced glucose uptake. For therapeutic uses, the compositions or agents identified using the methods disclosed herein may be administered systemically, for example, formulated in a pharmaceutically acceptable buffer such as physiological saline. Preferable routes of administration include, for example, oral, subcutaneous, intravenous, intraperitoneally, intramuscular, or intradermal injections, which provide continuous, sustained levels of the drug in the patient. Treatment of human patients or other animals will be carried out using a therapeutically effective amount of an identified compound in a physiologically acceptable carrier. Suitable carriers and their formulation are described, for example, in Remington's Pharmaceutical Sciences by E. W. Martin. The amount of the active compound to be administered varies depending upon the manner of administration, the age and body weight of the patient, and with the type of disease and extensiveness of the disease Generally, amounts will be in the range of those used for other agents used in the treatment of diabetes.

### EXPERIMENTAL METHODS

### Materials

Rat skeletal muscle cells L6 were obtained from The American Type Culture Collection (ATCC). Human neuroblastoma cells, SH-SY5Y, were obtained from European Collection of Animal cell cultures (ECACC). Human skeletal muscle cells (hSkMC) were purchased from PromoCell, Germany. The insulin-secreting cell line INS-1, isolated from rat insulinoma (Asfari et al. (1992) Endocrinology 130: 167-178) was obtained from Prof. C. B. Wollheim, University Medical Center, Geneva, Switzerland.

Bovine insulin, Dulbecco's Modified Eagle's Medium (DMEM), RPMI 1640 medium, Phosphate Buffered Saline (PBS), HEPES, pyruvate, 2-mercaptoethanol, Foetal Bovine Serum (FBS), penicillin and streptomycin (PEST) were purchased from Gibco Laboratories. Growth medium for hSkMC was purchased from PromoCell, Germany. Wortmannin and PD98059 were obtained from Alexis Biochemicais, San Diego, Calif., USA. U0126 was purchased from Promega Corporation, Madison, Wis., U.S.A. Tissue culture plates were purchased from Costar, U.S.A. Bovine Serum Albumin (BSA), dexamethasone, IBMX, Tris/HCl, EGTA, MgCl₂, ATP, microcystin, sodium orthovanadate, sodium betaglycerophosphate, NaF, MOPS, DTT and protease inhibitors were obtained from Sigma, USA.

U-¹⁴C-glucose, ³H-2-deoxy-glucose, U-¹⁴C-palmitate and ³³P-ATP were from Du Pont NEN, Medical Scandinavia. Sweden. Whatman No. 1 filter papers and p81 phosphocellulose filter papers from Kebo Lab., Sweden, hyamine hydroxide from ICN, USA.

Anti-PKBα antibody pre-coupled to protein A beads was purchased from Upstate, USA, AG10 anti-phosphotyrosine antibody were bought from Santa Cruz Biotechnology, INC, USA, and goat anti-mouse horseradish peroxidase-coupled secondary antibody from DAKO, Denmark. The protein concentrations in cell lysates were determined using a protein determination kit from Pierce, USA. Rat insulin was determined with an ELISA from Mercodia AB, Uppsala, Sweden.

### Cell Cultures

Rat L6 myoblasts were grown in culture flasks in DMEM containing 10% FBS and 2% PEST. To initiate differentiation, the media of sub-confluent cell cultures were replaced with DMEM supplemented with 1% FCS and 0.3 μM insulin as described in Klip, A. et al. (1984) Am. J. Physiol. 247: E291-E296 and Walker, P. S. et al. (1989) J. Biol. Chem. 264: 6587-6595. SHSY5Y cells were grown in DMEM containing 10% FBS. INS-1 cells were grown in RPMI 1640 medium supplemented with 10 mM HEPES, 1 mM pyruvate, 50 μM 2-mercaptoethanol and 5% FBS.

### Determination of Glucose Uptake Rate

Glucose uptake was determined as described by Hundal et al. (1994) Biochem. J. 297: 289-295. Briefly, after incubation with insulin for one hour, cell monolayers were rinsed with glucose-free PBS. Glucose uptake was quantified by incubating the cells in the presence of 1 μCi/ml ³H-2-deoxy-glucose in PBS for 8 min. Uptake of 2-deoxy-glucose was terminated by rapidly aspirating the medium, followed by two successive washes of cell monolayers with ice cold PBS. The cells were lysed in 0.5 M NaOH, followed by liquid scintillation counting. Rates of transport were normalized for protein content in each well.

### Determination of Glucose and Palmitic Acid Oxidation Rates by ¹⁴CO₂ Trapping

The principle of the used glucose/free fatty acid, (FFA) oxidation assay is based on the fact that one of the final products along metabolic pathways of these two substrates is carbon dioxide. Since the substrates are uniformly ¹⁴C labeled, the radioactivity in carbon dioxide trap is a direct measure of metabolic activity in studied cells (Rodbell, M. (1964) J. Biol. Chem. 239: 375-380).

As further described in International Patent Application No. PCT/SE00/02680, filed Dec. 27, 2000, cells were cultivated until sub-confluence in T-25 Costar flasks. Prior to the experiment, the cells were deprived of serum for 6 hours in DMEM medium containing 5 mM glucose. 3 ml of medium supplemented with (U-¹⁴C)-glucose or (U¹⁴C)-palmitic acid (0.2 μCi/ml) were added to each flask. A filter paper (1.5×5.5 cm) soaked in hyamine solution was rolled up, blotted on a paper towel to remove excess of fluid, and placed carefully into the tube of the device described in International Patent Application No. PCT/SE00/02680. The tube was mounted in the flasks, the screw caps were tightened and cells were incubated at 37° C. for a suitable time period. The reaction was terminated by adding sulfuric acid to the culture medium and the cells were incubated for additional 60 min. The filter paper was removed, cut into small pieces and transferred to scintillation vials. Methanol (0.2 ml) was added to each vial to increase the solubility of hyamine-CO₂ in the scintillation fluid. Finally the radioactivity was measured. The remaining cells were washed briefly with ice cold PBS, solubilized with 1 M KOH and the protein content was determined by the Bradford method (Bradford, M. M. (1976) Anal. Biochem. 72: 248-254).

### EXAMPLES

### Example 1

### Glucose Uptake Rate in L6 Cells

Glucose uptake rate in L6 cells, in the presence of 5 mM glucose, was determined. The cells were incubated in serum-free medium supplemented with U0126 (10 μM), PD98059 (10 μM) or wortmannin (100 nM) for 20 h. Insulin (176 nM) was added 1 h before the determination of glucose uptake rate. Glucose uptake was determined as described above. The results (FIG. 2) indicate that U0126 and insulin increased glucose uptake in L6 cells. When U0126 and insulin were incubated together there was no further increase compared to U0126 alone. Wortmannin inhibited the effect of U0126.

### Example 2

### Glucose Uptake Rate in Human Skeletal Muscle Cells

Glucose uptake rate in human skeletal muscle cells (hSkMC), in the presence of 5 mM glucose, was determined. The cells were incubated in serum-free medium (PromoCell) for 2 h. Insulin (176 nM) or U0126 (10 μM) were added 30 min before determination of glucose uptake rate. The results (FIG. 3) indicate that U0126 and insulin increase glucose uptake in hSkMC.

### Example 3

### Glucose Uptake Rate in SH-SY5Y Cells

Glucose uptake rate in SH-SY5Y cells in the presence of 5 mM glucose was determined. The cells were incubated in serum-free medium over night. Approximately 20 h later U0126 (10 μM), wortmannin (100 nM) or insulin (176 nM) were added, 1 h before the determination of glucose uptake rate. The results (FIG. 4) indicate that U0126 and insulin increased glucose uptake in SH-SY5Y cells Wortmannin inhibited the effect of U0126 and insulin.

### Example 4

### Glucose Oxidation in L6 Cells

Glucose oxidation in L6 cells in the presence of 5 mM glucose was determined as described in “Experimental Methods”. One hour prior to determination of glucose oxidation, insulin or U0126 was added to a concentration of 176 nM or 10 μM, respectively. The results (FIG. 5) indicate that U0126 and insulin increased glucose oxidation in L6 cells. A small increase was found when U0126 and insulin were incubated together.

### Example 5

### Palmitate Oxidation in L6 Cells

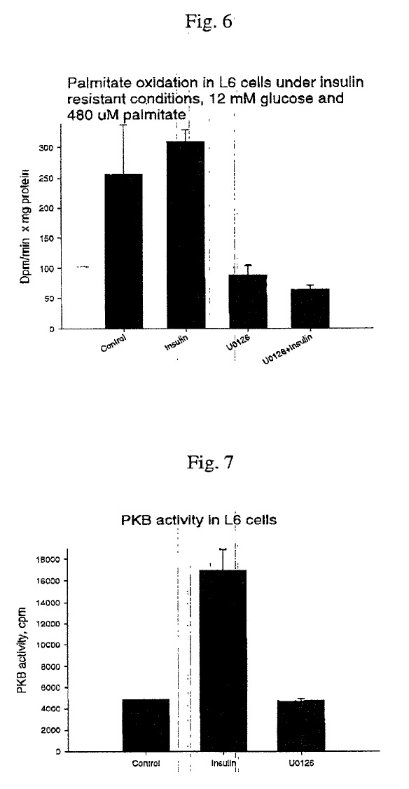

In a cell system, insulin resistance can be provoked experimentally by raising the concentrations of glucose, fat or both in the medium, or by treating the cells with the cytokine Tumor Necrosis Factor alpha (TNF alpha) before insulin stimulation. To achieve insulin resistance, L6 skeletal muscle cells were incubated in serum-free medium supplemented with 12 mM glucose and 480 μM palmitate bound to BSA for 20 hours. One hour prior lo determination of palmitate oxidation, insulin or U0126 was added to a concentration of 176 nM or 10 μM, respectively. Palmitate oxidation was determined as described in “Experimental methods”. Palmitate oxidation in cells is indicative of the use of fatty acids as energy and is reversed to glucose oxidation. Insulin regulates this process by increasing glucose oxidation and decreasing fat oxidation in normal cells without insulin resistance. The results (FIG. 6) indicate that U0126, in contrast to insulin, decreased palmitate oxidation during insulin resistant conditions. Both U0126 and insulin decreased palmitate oxidation in the presence of 5 mM glucose (data not shown).

### Example 6

### PKB Activity in L6 Cells

For the determination of PKB activity, differentiated L6 cells were starved with serum-free medium for 18 h and incubated with U0126 and insulin for 10 min. After the incubation the cells were washed with ice-cold serum-free medium prior to lysis with a buffer comprising 50 mM Tris/HCl, 0.1 mM EGTA, 10 mM MgCl₂, 100 μM ATP, 250 nM microcystin, 1 mM sodium orthovanadate, 10 mM sodium betaglycerophosphate, 40 mM NaF and protease inhibitors. Lysates were pre-cleared by centrifugation at 14,000 rpm for 10 minutes at 4° C. and stored at −70° C. until analysis. PKB was immunoprecipitated from cell lysate equivalent to 100 μg protein using a slurry of anti-PKBα antibody pre-coupled to protein A beads (equivalent to 200 ng antibody per immunoprecipitation). Immunoprecipitation occurred for 18 hours at 4° C. and beads were washed three times with buffer A containing 500 mM NaCl, once with lysis buffer alone and once with assay dilution buffer comprising 20 mM MOPS, 1 mM sodium orthovanadate, 25 mM sodium betaglycerophosphate and 1 mM DTT. PKB activity was assayed in “buffer A” containing 2-5 μCi ³³P-ATP in the presence of 50 μM Crosstide Peptide substrate, a peptide with the amino acid composition GRPRTSSFAEG. Peptide phosphorylation reactions were terminated by spotting 40 μl of 50 μl the reaction mixtures into p81 phosphocellulose filter papers, followed by washing with 0.75% phosphoric acid. Radioactivity was determined by liquid scintillation spectrometry. The results (FIG. 7) indicate that insulin increased PKB activity three-fold in the cells while U0126 had no effect.

### Example 7

### Insulin Secretion in INS-1 Cells

Insulin secretion in INS-1 cells was determined. INS-1 cells were seeded in 24-well plates. Thirty minutes before assay the glucose concentration was decreased to 2.8 mM. After the incubation in low glucose medium, glucose (up to 16 mM) or U0126 (10 μM) were added. After 24 hours, samples were collected and analyzed in the rat insulin ELISA. The results (FIG. 8) indicate that U0126 increased insulin secretion in INS-1.

1. A method for identifying an agent useful for the treatment or prevention of a medical condition related to reduced cellular uptake of glucose, said method comprising the steps of:
(i) identifying a target polypeptide interacting with the compound U0126;
(ii) contacting a candidate agent with the said target polypeptide; and
(iii) determining whether said candidate agent modulates the biological activities of the said target polypeptide, such modulation being indicative for an agent useful for the treatment or prevention of a medical condition related to reduced cellular uptake of glucose.

2. The method according to claim 1 wherein step (i) comprises identifying a target polypeptide binding to the compound U0126.

3. The method according to claim 1 or 2 wherein the said modulation in step (iii) comprises increasing cellular uptake of glucose.

4. The method according to any one of claims 1 to 3 wherein the said medical condition related to reduced cellular uptake of glucose is type II diabetes.

5. The method according to any one of claims 1 to 4 wherein the said target polypeptide is phosphoinositide 3-kinase.

6. The method according to any one of claims 1 to 5, comprising comparing the effect of the candidate agent with the effect of the compound U0126, a similar effect being indicative of an agent useful for the treatment or prevention of a medical condition related to reduced cellular uptake of glucose.
